SEQUENCE LISTING (3 sequences)

SEQ ID NO: 1 - protein, 228 aa, Homo sapiens
ETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 2 - protein, 112 aa, Homo sapiens
VGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCE

SEQ ID NO: 3 - protein, 18 aa, Homo sapiens
ERKALQTEMARIKKWLTF

(19) United States
(12) Patent Application Publication
(10) Pub. No.: US 2023/0241165 A1
(43) Pub. Date: Aug. 3, 2023

(54) MANNOSE-BINDING LECTIN FOR TREATMENT OR PROPHYLAXIS OF INFECTIOUS DISEASES

(71) Applicant: ESS HOLDING GmbH, Steyr (AT)

(72) Inventors: Alireza ESLAMIAN, Steyr (AT); Martin SCHIFKO, Steyr (AT)

(21) Appl. No.: 17/923,907

(22) PCT Filed: May 7, 2021
(86) PCT No.: PCT/AT2021/060161

§ 371 Date: Nov. 7, 2022

(30) Foreign Application Priority Data
May 7, 2020 (AT) A50393/2020

(51) Int. Cl. A61K 38/17; A61K 38/16; A61K 9/00; A61P 31/14; A61K 9/68

(52) U.S. Cl. CPC A61K 38/1732; A61K 38/168; A61K 9/0014; A61P 31/14; A61K 9/006; A61K 9/0058; A61K 9/0056; A61K 9/0043; A61K 9/0048

(57) ABSTRACT

The present invention is, inter alia, directed towards mannose-binding lectin and compositions comprising mannose-binding lectin for use in treatment or prophylaxis, towards compositions comprising mannose-binding lectin and towards the use of mannose-binding lectin and said compositions. Mannose-binding lectin, or portions thereof are applied locally according to the invention. MBL is binding to a pathogen to prevent it from binding to a host cell by blocking the binding sites of the pathogen. As a result, infection or transmission of disease can be avoided.

### TECHNICAL FIELD

The present invention is, inter alia, directed towards mannose-binding lectin and compositions comprising mannose-binding lectin for use in treatment or prophylaxis, towards compositions comprising mannose-binding lectin and towards the use of mannose-binding lectin and said compositions.

### BACKGROUND

Many diseases are caused by pathogens, such as viruses, bacteria or fungi. Pathogens use different entry points to enter the body of their host e.g. wounds in the skin or mucous membranes. Pathogens transmitted by droplets or through airborne transmission enter the respiratory system and get inside the body through the nose, mouth or eye surfaces. The aerosols and droplets spread by speaking, laughing, sneezing or coughing. This respiratory route is common for pathogens causing respiratory diseases, such as influenza. Some pathogens also survive on surfaces and get transmitted through contaminated surfaces or by direct contact.

However, not every contact with a pathogen leads to an outbreak of a disease. Pathogens differ in their infectivity; this is the ability to infect a host. The smallest quantity of infectious material that regularly produces infections is called minimal infective dose. In theory one single pathogen is enough to infect a host but it has been shown that the dose of pathogens the body is confronted with, is an important factor, not only for the question, if the host gets infected but also for the severity of symptoms.

Host factors, such as appropriateness of the immune response, also contribute to the virulence of a pathogen. In many species, there are two major subsystems of the immune system: the innate immune system and the adaptive immune system. Some defence mechanisms evolved in ancient eukaryotes and were passed on their modern descendants.

In mammals, mannose-binding lectin (MBL) is an important element of the first response of the host and also in the progression of the infection. MBL (also called mannose- or mannan-binding protein, MBP1) is a member of the collectin family that belongs to the upper group of C-type lectin receptors. MBL is a serum lectin that binds to mannose, N-acetylglucosamine (NAG)-containing carbohydrates, and various other carbohydrates that are present on the surface of many microbial pathogens.

Human MBL is a polymeric protein assembled from three or more 32 kDa monomers. Each monomer has an N-terminal cysteine rich region, a collagen-like gly-X-Y region, a coiled-coil neck region and a carbohydrate recognition domain. The assembly of the higher molecular weight polymers begins with formation of trimers of the 32 kDa monomer, these trimers then self-assembly into higher molecular weight polymers of three to six sets of trimers. Human MBL complexes consisting of five to six repeats of the functional MBL trimer are potent activators of the complement system via this lectin pathway. MBL is a key component in opsonization of microbial pathogens. Opsonization is a process by which the binding of proteins marks target cells for ingestion and destruction by phagocyte cells, such as macrophages and neutrophils. Via the lectin pathway specialised proteins i.e., MASP-1 (Mannan-binding lectin Associated Serine Protease) and MASP-2, interact with pathogen bound MBL and activate the complement system. MBL thus inactivates pathogens and supports the recognition and destruction of a target cell, e.g. a pathogen or pathogen infected cell.

Human MBL is produced mainly in the liver. The median MBL concentration varies among people in different countries. In an Iranian study, children under the age of 6 years had a median MBL concentration of 3.960 μg/ml, while in China children under the age of 6 years had a median MBL concentration of 2.536 μg/ml.

The Iranian study also tested adults that had a median MBL concentration of 1.858 μg/ml. In a study conducted in Switzerland children and adolescents under the age of 16 years had a median MBL concentration of 1.960 μg/ml, while adults had a median MBL concentration of 1.130 μg/ml. The median MBL concentration in Japan was higher with young people under the age of 20 having a median MBL concentration of 2.330 μg/ml, while adults still had a median MBL concentration of 1.280 μg/ml.

The highest level of MBL was reported in a published study, where newborns at the age of 1 month had an MBL concentration of 8.49 μg/ml, and adults had a median MBL concentration of 4.02 μg/ml. The high blood levels of MBL can be explained by a traditional diet that has a high MBL-content.

The MBL gene (MBL2) is located on Chromosome 10 and comprises four exons. Polymorphisms in the MBL2 structural gene are quite frequent with 30% of normal blood donors are heterozygous for structural gene mutations and a further 8% are homozygous or have double mutations. Allelic variants causing low MBL levels are found in most ethnic groups.

MBL deficiency is associated with infections such as tuberculosis and a higher prevalence for infections with Pseudomonas aeruginosa. Low levels of MBL were also found in patients with autoimmune diseases such as rheumatoid arthritis, systemic lupus erythematosus or Sjörgen's syndrome.

Higher levels of MBL correlate with a lower incidence of infectious diseases and have been found to be associated with a lower risk for heart attacks and heart disease in general. Some studies suggested supplementation of plasma-derived MBL via injection, e.g. “Mannan-binding Lectin (MBL) Production From Human Plasma, I Laursen, Biochem Soc Trans. 2003 August; 31(Pt 4):758-62” and “The pharmacokinetic profile of plasma-derived mannan-binding lectin in healthy adult volunteers and patients with Staphylococcus aureus septicaemia, Peter Bang et al., Scandinavian Journal of Infectious Diseases, 40:1, 44-48”. The mean residence time of administered MBL in healthy adults was 82 hours.

However, these treatments are quite complex and not suitable for widespread use.

While MBL is found in mammals, other mannose-binding lectins, similar lectins or lectin-like proteins play a key role in pathogen recognition in other classes of life, such as animals, particularly fish or crustaceans, and fungi or plants. Consequently, mannose-binding lectin is present in various foods. Mannose-binding lectin containing foods and plants are, among others, yam tuber, banana, cyanobacteria and algae, mistletoe, jackfruit seeds, tomato, garlic, curcuma and wild garlic. However, mannose-binding lectins have not been used in treatment or prophylaxis until now.

### Problem

The objective of this invention is to prevent infections, particularly infections transmitted by the respiratory route or via the eye. A further objective is the treatment of diseases at the point of entry into the body.

### SUMMARY OF THE INVENTION

The problem is solved by a mannose-binding lectin, or a portion thereof, for use according to claim 1.

Infections can be prevented by mannose-binding lectin for use in prophylaxis of infectious diseases, wherein the mannose-binding lectin is applied locally. A suitable treatment of infectious diseases can be provided by mannose-binding lectin for use in the treatment of infectious diseases, wherein the mannose-binding lectin is applied locally.

The local application of mannose-binding lectin increases the concentration of mannose-binding lectin at the point of entry of a pathogen. Pathogens that are present at the application site are bound by the mannose-binding lectin. This can prevent the pathogens from entering the body, as the necessary binding sites are blocked by the mannose-binding lectin. MBL can also help prevent the spread of a pathogen by an infected person.

While increasing the MBL blood-level can support the immune system once a virus was detected by the immune system, the virus is only recognized after connecting to the cell membrane and proliferation has already started. This causes an important time lag between entry of the virus into the body and reaction of the immune system, since viruses use many mechanisms of hiding from the immune system. Accordingly, by making mannose-binding lectin available at the point of entry, pathogens are bound to said mannose-binding lectin before they can bind to membrane cells of the host. This is important, because after binding to host cells, viruses can shield from the immune system.

Optional features further improving the invention are summarised in the dependent claims.

A major entry point for infectious diseases is the mucosa of the respiratory system or via the eye. By local application to the respiratory system or via the eye these infections can be prevented. It is particularly useful, when mannose-binding lectin is applied orally, nasally, to the lung, or into the eyes. It is particularly advantageous, when the infectious disease is transmitted by the respiratory route or via the eye, and when the mannose-binding lectin is applied to the respiratory system or via the eye, particularly orally, nasally, to the lung, or into the eyes. In this case, by locally applying mannose-binding lectin, the entry into the body, as well as the spreading of the pathogen, e.g. the virus, can be reduced or even prevented.

As an example, SARS-CoV-2 uses sialic acid to hide from the immune systems even for several days while spreading in the respiratory system and getting transmitted to others. Further, an application in or to the eye can help to prevent infections.

Formulations for each route of administration are well known in the art. Particularly useful is mannose-binding lectin applied by means of a chewing gum, ice cream, lozenge, toothpaste, mouth wash and/or gargling solution, mouth spray, nasal spray, nasal drops, nasal cream, eye drops, eye cream or ointment, inhalation, particularly an aerosol spray. It is particularly preferred, when the mannose-binding lectin is kept at the place of application for a longer time period. This can be achieved by suitable pharmaceutical carriers.

In some embodiments the mannose-binding lectin can be applied to a face mask. In one preferred method for protection against spreading of diseases mannose-binding lectin, e.g. human MBL or BanLec, is applied to a face mask. In certain embodiments an aerosol spray is applied to the face mask just before use or while already in use.

When species-specific mannose-binding lectin, e.g. MBL is used, the pathogen bound by MBL is detected by the immune system and the immune response is initiated. As a result, the time lag between appearance of the pathogen and reaction of the immune system can be reduced dramatically.

For human application, a particular suitable mannose-binding lectin is human MBL. For example, this can be provided as recombinant human MBL. By local application of human MBL the pathogen is not only prevented from binding to the cells of the mucosa, but it is also marked for detection by the immune system. As a result, mechanisms such as phagocytosis and the innate complement pathway can readily be activated. Further, human MBL can be adsorbed at the site of application and locally support the immune response of the cells.

When hMBL is absorbed, it can help to compensate an unfavorable relation of MBL and sugar consumption. hMBL can be used as prophylaxis in diabetes 2 risk candidates. Further hMBL works as prophylaxis for Alzheimer's disease and as prophylaxis for HIV.

However, an immune response is not always intended, and the invention is particularly directed at MBL for binding to a pathogen to prevent it from binding to a host cell by blocking the binding sites of the pathogen.

In a preferred embodiment, MBL is hMBL corresponding to

```
(SEQ ID NO: 1)

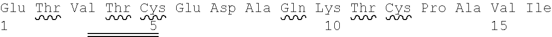

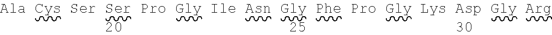


Asp Gln Thr Lys Gly Glu Lys Gly Glu Pro Gly Gln Gly Leu Arg Gly
                35 40 45
Leu Gln Gly Pro Pro Gly Lys Leu Gly Pro Pro Gly Asn Pro Gly Pro
          50 55 60
Ser Gly Ser Pro Gly Pro Lys Gly Gln Lys Gly Asp Pro Gly Lys Ser
65 70 75 80
Pro Asp Gly Asp Ser Ser Leu Ala Ala Ser Glu Arg Lys Ala Leu Gln
                                        85 90 95
Thr Glu Met Ala Arg Ile Lys Lys Trp Leu Thr Phe Ser Leu Gly Lys
                              100 105 110
Gln Val Gly Asn Lys Phe Phe Leu Thr Asn Gly Glu Ile Met Thr Phe
                        115 120 125
Glu Lys Val Lys Ala Leu Cys Val Lys Phe Gln Ala Ser Val Ala Thr
                    130 135 140
Pro Arg Asn Ala Ala Glu Asn Gly Ala Ile Gln Asn Leu Ile Lys Glu
          145 150 155
Glu Ala Phe Leu Gly Ile Thr Asp Glu Lys Thr Glu Gly Gln Phe Val
160 165 170 175
Asp Leu Thr Gly Asn Arg Leu Thr Tyr Thr Asn Trp Asn Glu Gly Glu
                              180 185 190
Pro Asn Asn Ala Gly Ser Asp Glu Asp Cys Val Leu Leu Leu Lys Asn.
                        195 200 205
```

To improve binding of the hMBL, a method is provided wherein the portion of the hMBL corresponding to

```
(SEQ ID NO: 2)
Val Gly Asn Lys Phe Phe Leu Thr Asn Gly Glu Ile Met Thr
1 5 10
Phe Glu Lys Val Lys Ala Leu Cys Val Lys Phe Gln Ala Ser
15 20 25
Val Ala Thr Pro Arg Asn Ala Ala Glu Asn Gly Ala Ile Gln
        30 35 40
Asn Leu Ile Lys Glu Glu Ala Phe Leu Gly Ile Thr Asp Glu
                45 50 55
Lys Thr Glu Gly Gln Phe Val Asp Leu Thr Gly Asn Arg Leu
                          60 65 70
Thr Tyr Thr Asn Trp Asn Glu Gly Glu Pro Asn Asn Ala Gly
                                  75 80
Ser Asp Glu Asp Cys Val Leu Leu Leu Lys Asn Gly Gln Trp
85 90 95
Asn Asp Val Pro Cys Ser Thr Ser His Leu Ala Val Cys Glu,
        100 105 110
```

particularly corresponding to

(SEQ ID NO: 3)
Glu Arg Lys Ala Leu Gln Thr Glu Met Ala Arg Ile
1 5 10
Lys Lys Trp Leu Thr Phe.
15

A higher affinity can be achieved, if portions of MBL are used, that comprise the glycoprotein-binding site. For hMBL the affinity is improved, if the portion comprises amino acid residues 81 to 128 of hMBL (SEQ ID NO: 2), particularly amino acid residues 111 to 128 of hMBL (SEQ ID NO: 3).

However, the invention can also be carried out by applying a plant-derived mannose binding lectin or portions thereof. The effectiveness of plant lectins as inhibitors of coronaviruses has been shown for example in “Els Keyaerts et al., Antiviral Res. 2007 September; 75(3): 179-187”.

The plant-derived mannose-binding lectin can particularly be selected from the group of ACA (Allium cepa), APA (Allium porrum), ASA I (Allium sativum), ASA II (Allium sativum), AUA (Allium ursinum), ArtinM (Artocarpus heterophyllus), B7U6V0 (Zingiber officinalis), BanLec (Musa acuminate), ConA (Canavalia ensiformis), DB1 (Dioscorea batatas), LEA (Solanum lycopersicum), Morniga M II (Morus Nigra), Q1S2H7 (Curcuma zedoria) or GRFT/Griffithsin (Griffithsia). Particularly useful is BanLec or a portion thereof.

In addition, the application of an animal-derived or fungi-derived mannose-binding lectin is possible. This animal-derived mannose binding lectin can particularly be selected from the group of LvCTL1 (Litopenaeus vannamei), Pl-MBL (Pacifastacus leniusculus), PcLec4 (Pacifastacus clarkia), trout-MBL1 (Oncorhynchus mykiss), trout-MBL2 (Oncorhynchus mykiss) or AbMb (Agaricus bisporus).

In addition, the invention can also be carried out by applying portions of human, plant-derived, recombinant, or animal derived mannose-binding lectins where the portion of the mannose-binding lectin has maintained or enhanced affinity to mannose and/or, N-acetylglucosamine (NAG)-containing carbohydrates, and various other carbohydrates.

Whenever mannose-binding lectin is mentioned, human, plant-derived, recombinant or animal derived mannose-binding lectins or portions thereof, are meant and understood. In some cases, this can also mean a single molecule.

Mannose-binding lectin for use is particularly effective in infectious diseases transmitted by the respiratory route.

Mannose-binding lectin can be effective in the treatment or prophylaxis of infectious diseases caused by bacteria, fungi or by viruses, particularly by viruses having a viral envelope. Mannose-binding lectin is particularly effective in regulating the relation sugar to MBL inside human body.

Generally, the younger the person is the more MBL is produced by the liver, respectively, the older the person is the less MBL is produced. Diabetes type 2 typically arises with old people or people with an unbalanced diet. These people should compensate the diet to the MBL level or adapt the MBL level to the diet to compensate the danger of insulin resistance and many more immune illnesses.

The effect of mannose-binding lectin for use has been shown in the treatment and in the prophylaxis of Coronaviridae-induced diseases, particularly of SARS-CoV-2-induced diseases, e.g. COVID19. These findings are supported by observations: Median MBL concentration has been shown to be higher in children and children have been generally less affected by an infection with SARS-CoV-2. Additionally, for Finland, high levels of median MBL concentration have been reported and Finland was less affected by the pandemic outbreak of COVID-19.

For best results, mannose-binding lectin can be applied in an effective amount to prevent infectious diseases by binding pathogens before entering the body.

MBL blocks the binding sites of a pathogen, e.g. the spike protein of SARS-CoV-2. As a result, the pathogen is prevented from binding to a host cell. Any MBL or portion thereof, particularly as described before, can be used.

An easy way to apply the MBL is in form of eye drops, nasal spray, mouth spray, or as an aerosol spray for inhalation.

In a preferred embodiment MBL corresponding to SEQ ID NO: 2 or corresponding to SEQ ID NO: 3 is applied in form of an aerosol spray.

The positive effects of mannose-binding lectin can be observed, when mannose-binding lectin, depending to the administration method is applied in a concentration of 150 μg/ml and or more.

To support a continuous action mannose-binding lectin can be applied twice a day, particularly every 12 hours.

Further, mannose-binding lectin can be applied on demand, particularly shortly before an exposure to a pathogen, more particularly 0 to 60 minutes, particularly 5 to 60 minutes, before an exposure to a pathogen, e.g. a virus, particularly SARS-CoV-2. A person can apply mannose-binding lectin just before meeting others, for example before watching a concert. The mannose-binding lectin binds any pathogens at the point of application. This can protect the user from infection with a pathogen, but it also prevents the user from spreading pathogens the user is already infected with.

Particularly easy to handle is mannose-binding lectin in the form of drops. These can be applied into eyes, nose and mouth. An effective way of preventing transmission of pathogens is mannose-binding lectin in an aerosol spray for inhalation. In addition, or alternatively MBL can be applied to a face mask worn by a person.

Mannose-binding lectin depending to the administration method can be applied for example, Every 3 hours with almost 250 μg/ml concentration through mouth or every 6 hours with 150 μg/ml through eyes, nose or inhalation.

Another aspect of the invention is a composition for use in prophylaxis and/or treatment of infectious diseases. A composition according to the invention comprises mannose-binding lectin for local application and/or for increase of the local concentration of mannose-binding lectin.

The composition is particularly useful in infectious diseases transmitted by the respiratory route or via the eye, wherein the composition comprises mannose-binding lectin, particularly as described above, and wherein the composition is adapted for local application to the respiratory system or to the eye.

Such a composition can be applied orally, nasally, to the lung, or in the eye. Particularly useful is a composition that is adapted for application of mannose-binding lectin to lung, throat, mouth, nose or eyes.

The application is particularly easy, targeted and effective, when the composition is a chewing gum, ice cream, lozenge, toothpaste, mouth wash and/or gargling solution, nasal spray, nasal drops, nasal cream, eye drops, eye cream or ointment, inhalation, particularly an aerosol-spray, or as spray to be added to a face mask when or before using it.

Suitable compositions can comprise mannose-binding lectin in a concentration of 0.1 to 0.5 wt % of the composition.

An easy and effective application can be achieved, when the composition is applied twice a day, more particularly every 12 hours.

However, the composition can also be applied on demand, for example depending to the administration method every 3 hours with almost 250 μg/ml concentration through mouth or every 6 hours with 150 μg/ml through eyes, nose or inhalation as described for the use of mannose-binding lectin. The application of the composition is particularly easy to handle when the composition is applied as liquid drops, as it can be used for application to eyes, mouth and nose.

Such compositions are particular suitable for prophylaxis and/or treatment of infectious diseases caused by viruses, particularly viruses having a viral envelope, bacteria or fungi.

Good results have been achieved in the prevention and treatment of Coronaviridae-induced diseases, particularly of SARS-CoV-2-induced diseases, e.g. COVID19. Particularly effective in this regard is human MBL or a portion thereof, or BanLec or a portion thereof.

The effectiveness of the composition can be enhanced, when the composition comprises an anti-inflammatory agent, particularly benzydamine or betamethasone.

For infectious diseases that come with inflammatory symptoms, e.g. COVID19, a combination preparation comprising mannose-binding lectin, particularly as described above, more particularly human MBL, and an anti-inflammatory agent, particularly corticosteroids, more particularly betamethasone, is highly useful.

Corticosteroids like betamethasone can act through nongenomic and genomic pathways. Glucocorticoids inhibit neutrophil apoptosis, and inhibit NF-Kappa B and other inflammatory transcription factors. They also inhibit phospholipase A2, leading to decreased formation of arachidonic acid derivatives. In addition, glucocorticoids promote anti-inflammatory genes like interleukin-10. Thus, by the combination of MBL and corticosteroids the immune response can be altered such that the pathogen is targeted more effectively.

To further support the immune system and to enhance the effectiveness of the composition an antimicrobial agent may be comprised, particularly an agent selected of the group of cetylpyridinium, chlorhexidine, hexetidine, hydrogen peroxide, nystatin, tetracycline, triclosan, or essential oils, more particularly eucalyptus oil.

The treatment of infectious diseases causing local irritations can be supported when the composition comprises a local anaesthetic, particularly benzydamine, benzocaine or lidocaine.

Further, particularly for the treatment of persisting chesty cough, e.g. COVID19, the composition may comprise a cough suppressant, particularly dextromethorphan.

Easy and effective application and prolonged release can be achieved, when the composition is a chewing gum comprising 25 to 35 wt % of a gum base and 0.1 to 0.5 wt % mannose-binding lectin.

For easy and regular application, it is particularly useful, when the composition is a toothpaste comprising at least 50 wt % abrasives, 20 to 42 wt % water and 0.1 to 0.5 wt % mannose-binding lectin.

For situations where infections might easily be transmitted, such as use of public transport or in crowded places, the composition can easily be used without attracting attention, when the composition is a lozenge comprising eucalyptus oil and 0.1 to 0.5 wt % mannose-binding lectin.

Another suitable composition for easy application are nasal and oral drops comprising water and 0.1 to 0.5 wt % mannose-binding lectin.

Another suitable composition for easy application is nasal and oral spray comprising water and 0.1 to 0.5 wt % mannose-binding lectin.

Another embodiment of the invention is a method for treatment and/or prevention of an infectious disease in an individual comprising locally administering to said individual mannose-binding lectin and a pharmaceutically acceptable carrier.

The method is particularly useful when the individual is a human, particularly a human with MBL-deficiency.

It has been shown that the method is suitable when the infectious disease is a virus-induced disease, particularly a Coronaviridae-induced disease, more particularly a SARS-CoV-2-induced disease, e.g. COVID19.

For infectious diseases transmitted by the respiratory route, such as COVID19, it is particularly useful, when the method includes administering mannose-binding lectin, particularly human MBL, to the respiratory system.

A suitable method can include administering mannose-binding lectin to mouth, nose, lung or eye. A particularly easy and effective administration can be achieved by a composition as described above, e.g. via a chewing gum, ice cream, lozenge, toothpaste, mouth wash and/or gargling solution, nasal spray, nasal drops, nasal cream, eye drops, eye cream or ointment, inhalation, or as spray to be added to a face mask when using.

A further embodiment of the invention is the use of mannose-binding lectin in a medicament for local treatment and/or prevention of an infectious disease transmitted by the respiratory route.

The use of mannose-binding lectin, particularly human MBL or BanLec, is particularly useful for treatment and/or prevention of Coronaviridae-induced diseases, particularly by SARS-CoV-2, e.g. COVID-19.

The local administration of human MBL helps the immune system to tackle pathogens and prevents the occurrence of infectious diseases. Human MBL acts directly by binding pathogens at the location of application, as well as indirectly by absorption through mucosa and skin. However, the invention is directed to any MBL or portion thereof that is binding to a pathogen and preventing it from entering the host cell by blocking the binding sites of the pathogen.

The invention is described by the following detailed description without limiting the scope of the claims.

### DETAILED DESCRIPTION

The term “comprising” encompasses “including” as well as “consisting” e.g. a composition “comprising” X may consist exclusively of X or may include something additional e.g. X+Y. In some implementations, the term “comprising” refers to the inclusion of MBL, as well as inclusion of other active agents. However, pharmaceutically acceptable carriers of active agents and other compounds may be included which are for stabilizing, preserving, etc. the composition. These are known by the skilled person in the pharmaceutical industry.

The local effect of MBL has been described for example in “Mitchell, C. et al. Antiviral Lectins: Selective Inhibitors of Viral Entry. Antiviral Res. 2017 June; 142: 37-54.”

The respiratory system comprises the nasal cavity, mouth, pharynx, larynx, trachea and lungs.

Infectious diseases of the respiratory system, but also some other diseases, are transmitted by the respiratory route. This includes airborne transmission by aerosols. The aerosols are predominantly dry and stay in the air for a long time. For example, they are released, when an infective individual is breathing or speaking. An example of an airborne transmitted disease is COVID19. The respiratory route also includes transmission by droplets. These are larger particles that contain water. As they are heavier than aerosols they stay in the air for a shorter time. However, they can remain on surfaces for a longer time and cause transmission of a disease by contact with the contaminated surface. The droplets are usually released, when an infective individual is coughing or sneezing. An example of a disease transmitted by droplets is influenza, but also COVID19.

Lectins from various sources have been shown to exhibit potent antiviral properties by inhibiting infection of clinically important viral pathogens. Based on prior studies on coronaviruses such as SARS-CoV and MERS-CoV, mannose-specific plant lectins can be used to investigate antiviral properties of the novel coronavirus SARS-CoV-2, the virus that causes COVID-19.

Some plant-derived and animal-derived mannose-binding lectins have been described in more detail:

BanLec has been described in detail in “Banana Lectin: A Brief Review, Molecules. 2014 November; 19(11): 18817-18827.”

Griffithsin is derived from Griffithsia spp. It has been described in “The need for ocular protection for health care workers during SARS-CoV-2 outbreak and a hypothesis for a potential personal protective equipment, Lixiang Wang and Yingping Deng, Front. Public Health, 12 Nov. 2020” and in “Griffithsin with a broad-spectrum antiviral activity by binding glycans in viral glycoprotein exhibits strong synergistic effect in combination with a Pan-Coronavirus fusion inhibitor targeting SARS-CoV-2 spike S2 subunit, Cai et al., Virol Sin., 2020 December; 35(6):857-860”. It also helps for ocular protection.

B7U6V0 has been described in detail in “https://www.uniprot.org/uniprot/B7U6V0”.

The mode of action for jackfruit has been described in “Effect of ArtinM on Human Blood Cells During Infection with Paracoccidioides brasiliensis, Luciana Pereira Ruas et al., Front. Microbiol., 4 May 2018”.

A lectin found in tomato plants, LEA, has been described in “Analysis of Sugar Chain-Binding Specificity of Tomato Lectin Using Lectin Blot: Recognition of High Mannose-Type N-glycans Produced by Plants and Yeast, Suguru Oguri, Glycoconj J 2005 November; 22(7-9):453-61”.

Garlic lectins bind to high mannose oligosaccharide chains according to “Garlic (Allium sativum) Lectins Bind to High Mannose Oligosaccharide Chains, Tarun Kanti Dam et al., The Journal of Biological Chemistry 273, 5528-5535”.

A lectin from Curcuma zedoaria Rosc, referred to as Q1S2H7, has be identified as Mannose-binding lectin, and has been described in “Mannose-binding lectin from Curcuma zedoaria Rosc, Tipthara, P, Biol. 50, 167-173 (2007)”.

Lectins in cyanobacteria and algae have been described in “Mannose-Specific Lectins from Marine Algae: Diverse Structural Scaffolds Associated to Common Virucidal and Anti-Cancer Properties, Annick Barre et al., Mar Drugs. 2019 Aug. 17(8):440.” and “Purification and Characterization of a new Lectin from the Red Marine Alga Hypnea Musciformis, Celso Shiniti Nagano et al., Protein and Peptide Letters 9(2):159-165 April 2002”.

Another MBL was identified in the Korean mistletoe, and described in “Concanavalin A and Mistletoe Lectin I Differentially Activate Cation Entry and Exocytosis in Human Neutrophils: Lectins May Activate Multiple Subtypes of Cation Channels, K Wenzel-Seifert et al., J Leukoc Biol. 1996 September; 60(3):345-55”.

MBL found in the rainbow trout has been described in “Molecular cloning and characterisation of two homologues of Mannose-Binding Lectin in rainbow trout, Konstantina Nikolakopoulou et al., Fish & Shellfish Immunology, Volume 21, Issue 3, September 2006, Pages 305-314”.

Also in shrimp MBL has been describes, for example in “A Novel C-Type Lectin from the Shrimp Litopenaeus vannamei Possesses Anti-White Spot Syndrome Virus Activity, Zhi-Ying Zhao et al., J Virol. 2009 January; 83(1): 347-356”.

A MBL found in salmon was described in “Identification of a pathogen-binding lectin in salmon serum, K. Vanya Ewart, Comparative Biochemistry and Physiology Part C: Pharmacology, Toxicology and Endocrinology, Volume 123, Issue 1, May 1999, Pages 9-15”.

MBL derived from crayfish was described in “An MBL-like protein may interfere with the activation of the proPO-system, an important innate immune reaction in invertebrates, Chenglin Wu, Immunobiology 218(2), February 2012”.

Also in Bluefin tuna, a MBL has been identified and described in “Isolation and Partial Characterisation of Immunoglobulin From Southern Bluefin Tuna Thunnus maccoyii Castelnau, M Watts et al., Fish Shellfish Immunol 2001 August; 11(6):491-503”.

Furthermore, in the carp family MBL has been described, e.g. in “The homologue of mannose-binding lectin in the carp family Cyprinidae is expressed at high level in spleen, and the deduced primary structure predicts affinity for galactose, Lars Vitved et al., Immunogenetics 51(11):955-64”.

Further, MBL has been described in fungi, for example in Agaricus bisporus, e.g. in “Orf239342 from the Mushroom Agaricus bisporus is a Mannose Binding Protein, Heni Rachmawati, Biochem Biophys Res Commun 2019 Jul. 12; 515(1):99-103”.

As proteins, it is assumed that lectins will not be able to be orally administered as they would not survive digestive enzymes. Antiviral lectins, due to their proteinaceous nature, have not been reported to be biologically-available following oral administration.

However, some reports suggest, that some lectins withstand the gastrointestinal passage, e.g. garlic lectin (“Dietary garlic (Allium sativum) lectins, ASA I and ASA II, are highly stable and immunogenic, Clement F et al., Int. Immunopharmacol. 2010; 10: 316-32”).

The stability of the garlic lectins, their ability to withstand the gastrointestinal passage, and their recognition by the immune system prove their effectiveness.

However, to directly tackle pathogens at the point of entry, the aim is to provide mannose-binding lectin in a way to keep it at the point of entry, e.g. in the mouth or the nose, for some time.

The administration of Banana Lectin (BanLec) has been discussed in “Molecular Engineering of a Banana Lectin that Inhibits HIV-1 Replication, Michael D. Swanson, University of Michigan”.

Oral administration of BanLec has been performed in mouse models without deleterious effects. One disadvantage of oral delivery is that it would likely require higher amounts of lectin compared to a vaginally administered form, and it would likely require daily use. Some people have questioned whether consuming bananas could prevent HIV-1 infection. This seems unlikely since a large number of bananas would likely be needed to receive sufficient amounts of BanLec, and that most of the lectin in bananas is bound to starches and may not be effective in that form. Therefore, bananas are not sufficient for oral administration of BanLec.

The role and expression of MBL in the mucosa was studied in “Role of mannose-binding lectin in intestinal homeostasis and fungal elimination, L Choteau, Mucosal Immunology Vol 9 No 3, May 2016”.

The translocation of lectins through the mucosa was studied earlier, e.g. in “Identification of intact peanut lectin in peripheral venous blood; Wang Q et al., Lancet. 1998; 352:1831”.

This has also been studied for the nasal mucosa. Nasal and oral administration have been described, e.g. in “Mucosal immunogenicity of plant lectins in mice, E C Lavelle, Immunology. 2000 January; 99(1): 30-37”.

The mucosal immunogenicity of plant lectins with different sugar specificities was investigated in mice. For this study Viscum album (mistletoe lectin 1; ML 1), Lycospersicum esculentum (tomato lectin; LEA), Phaseolus vulgaris (PHA), Triticum vulgaris (wheat germ agglutinin (WGA), Ulex europaeus I (UEA 1) were used. Following intranasal or oral administration, the systemic and mucosal antibody responses elicited were compared with those induced by a potent mucosal immunogen (cholera toxin) and a poorly immunogenic protein (ovalbumin; OVA). After three oral or intranasal doses of cholera toxin, high levels of specific serum antibodies were measured and specific IgA was detected in the serum, saliva, vaginal wash, nasal wash and gut wash of mice. Immunization with ovalbumin elicited low titres of serum IgG but specific IgA was not detected in mucosal secretions. Both oral and intranasal delivery of all five plant lectins investigated stimulated the production of specific serum IgG and IgA antibody after three intranasal or oral doses. Immunization with ML 1 induced high titres of serum IgG and IgA in addition to specific IgA in mucosal secretions. The response to orally delivered ML 1 was comparable to that induced by CT, although a 10-fold higher dose was administered. Immunization with LEA also induced high titres of serum IgG, particularly after intranasal delivery. Low specific IgA titres were also detected to LEA in mucosal secretions. Responses to PHA, WGA and UEA 1 were measured at a relatively low level in the serum, and little or no specific mucosal IgA was detected.

The role of lectin in host defence against microbial infections has been described in “Lectin in host defense against microbial infections, Shieh-Liang Hsieh, 2020”.

The effectiveness against coronaviruses was investigated, e.g. in “Plant lectins are potent inhibitors of coronaviruses by interfering with two targets in the viral replication cycle, Keyaerts E. et al., Antiviral Res. 2007 September; 75(3):179-87.”

Coronaviruses and their surface were described e.g. in “Identification of N-linked carbohydrates from severe acute respiratory syndrome (SARS) spike glycoprotein, Ritchie G. et al., Virology. 2010 Apr. 10; 399(2):257-69” or “Post-translational modifications of coronavirus proteins: roles and function, Fung, S. and Liu, D X., Future Virol. (2018) 13(6), 405-430.”

The use of lectins to increase immune response after oral or nasal administration has also be shown. The following references show that higher concentrations of MBL in mucosa and saliva increase the human immune response. Also, in mice the immune response was improved in some experiments following oral or intranasal administration.

A higher concentration of MBL in mucosa and salvia was found to increase immune response. This was shown in “Protective role of mouse MBL-C on intestinal mucosa during Shigella flexneri invasion, Da-Ming Zuo et al., Int Immunol. 2009 October; 21(10): 1125-1134.” These findings suggest that mMBL-C may protect host intestinal mucosa by directly binding to the bacteria.

The expression of MBL not only in the liver, but also in mucosa and the implications of this finding has been discussed in “Mannose-binding lectin and maladies of the bowel and liver, Daniel L Worthley et al., World J Gastroenterol. 2006 Oct. 28; 12(40): 6420-6428.” Although it is clear that the liver is the chief contributor to plasma MBL, mucosal MBL production is relevant in localized immune defence.

The role of MBL in the immune response in the mouth was investigated e.g. in “Salivary agglutinin is the major component in human saliva that modulates the lectin pathway of the complement system, Sabrina T G Gunput et al., Innate Immunity 2016, Vol. 22(4) 257-265” and “The salivary scavenger and agglutinin binds MBL and regulates the lectin pathway of complement in solution and on surfaces, Martin P. Reichhardt et al., Front. Immunol., 16 Jul. 2012 Vol 3, Art 205”.

Intranasal application has been studied e.g. in “Mistletoe lectins enhance immune responses to intranasally co-administered herpes simplex virus glycoprotein D2, E. C. Lavelle et al., Immunology 2002 107268-274.” It was found that specific IgA responses were also induced, when lectins were applied intranasally. It was demonstrated, that mistletoe lectins I, II and III improve the immune response of the mucosa.

As described before in “Mucosal immunogenicity of plant lectins in mice, E C Lavelle, Immunology. 2000 January; 99(1): 30-37”, is was found that oral and intranasal application of plant lectins stimulated the production of specific serum IgG and IgA antibody after three intranasal or oral doses. The response was comparable to a potent mucosal immunogenic substance. Low specific IgA titres were also detected, especially to LEA, in mucosal secretions.

The effectiveness of local application of lectins was also shown in “Adjuvant Effect of Garlic Lectins (Asa I and Asa Ii) on Mucosal Immunity Induction Following Intranasal Immunization with Ovalbumin Antigen, Siddanakoppalu N Pramod1+ and Yeldur P Venkatesh2, 2013 International Conference on Agriculture and Biotechnology IPCBEE vol. 60(2013)”. Garlic lectins, in particular ASA I, exhibited strong systemic responses by both intradermal and intranasal administration.

Mannose-binding lectin can be applied in a certain dose by administering a dosage unit comprising the mannose-binding lectin in a suitable concentration.

For example toothpaste has been shown to be an effective route of administration for enzymes and proteins e.g. in “A randomised clinical study to determine the effect of a toothpaste containing enzymes and proteins on plaque oral microbiome ecology, S. E. Adams et al., Sci Rep. 2017; 7:43344. Published 2017 Feb. 27”; a randomised clinical study to determine the effect of a toothpaste containing enzymes and proteins on plaque oral microbiome ecology. The results demonstrated that a toothpaste containing enzymes and proteins can augment natural salivary defenses to promote an overall community shift resulting in an increase in bacteria associated with gum health and a concomitant decrease in those associated with periodontal disease.

The present invention is illustrated by the following figures and examples, which do not limit the scope of the invention described in the claims.

### EXAMPLES

### Example 1: Concentration of MBL

The following table shows suitable doses for application of human MBL:

The following doses are safe, and can increase the concentration of human MBL with 1 μg/ml (table 1) or 2 μg/ml (table 2):

TABLE 1

| Human weight | kg | 100 | 90 | 80 | 70 | 60 | 50 | 40 | 30 | 20 |
|---|---|---|---|---|---|---|---|---|---|---|
| Administer | μg | 571 | 514 | 457 | 400 | 343 | 286 | 229 | 171 | 114 |
| twice a day | | | | | | | | | | |

TABLE 2

| Human weight | kg | 100 | 90 | 80 | 70 | 60 | 50 | 40 | 30 | 20 |
|---|---|---|---|---|---|---|---|---|---|---|
| Administer | μg | 1 143 | 1 029 | 914 | 800 | 686 | 571 | 457 | 343 | 229 |
| twice a day | | | | | | | | | | |

### Example 2: Chewing Gum

A chewing gum is prepared, wherein the basic ingredients are gum base, softener/plasticizer and MBL.

Other optional additives can be sweeteners, flavourings and colours. The person skilled in the art knows suitable compositions, e.g. from “Modern chewing gum, Mestres J, (2008) in Fritz, D (ed.). Formulation and Production of Chewing and Bubble Gum (2 ed.). Kennedy's Publications Ltd. pp. 47-73”.

Chewing gum can come in a variety of formats ranging from 1.4 to 6.9 g per piece, and products can be differentiated by the consumer's intent to form bubbles or the preference for sugar containing or sugarless products.

The ingredients for the chewing gum in this example are shown in table 3:

TABLE 3

| | wt % of the | |
|---|---|---|
| ingredients | composition | example and/or functionality |
| gum base | 25-35% | Three main components make up common gum bases: resin, |
| | | wax, and elastomer. |
| | | Resin, e.g. terpene, is the main chewable portion. Wax |
| | | softens the gum. |
| | | Elastomers add flexibility. |
| sucrose | 40-50% | Other sweeteners can be used, e.g. other carbohydrates, |
| | | such as dextrose, glucose or corn syrup. |
| | | Alternative sweeteners such as erythritol, isomalt, xylitol, |
| | | maltitol, mannitol, sorbitol, lactitol can also be used. |
| | | Amounts have to be adapted in line with potency. |
| glycerol | 2-15% | Moisturizer |
| softener/ | 1-2% | To soften gum by increasing flexibility and reducing |
| plasticizer | | brittleness by altering the glass transition temperature. |
| | | Quantities of this additives are altered in order to balance |
| | | processability and packaging speed. |
| | | Examples are lecithin, hydrogenated vegetable oils, glycerol |
| | | ester, lanolin, methyl ester, pentaerythritol ester, rice bran |
| | | wax, stearic acid, sodium and potassium stearates. |
| flavours | 1.5-3.0% | Peppermint and spearmint are the most popular flavours. |
| | | Food acids are implemented to provide a sour flavour (i.e. |
| | | citric, tartaric, malic, lactic, adipic, and fumaric acids). |
| colours | Variable | Natural or synthetic colours, appearance |
| coating/ | Variable | E.g. polyols, such as sorbitol, maltitol I/isomalt, mannitol; |
| humectant | | Polyols can also be implemented as humectants in form of |
| | | water absorbent powder dusting in order to maintain the |
| | | quality and extend the shelf life of the product. |
| mannose- | 0.1-0.5% | In this example BanLec was used; |
| binding | | The use of other mannose-binding lectins, e.g. human MBL, |
| lectin | | is possible. |

Recipe to prepare 100 g of MBL enriched chewing gum.

Ingredients:
30 g gum base
30 g corn syrup
37 g powdered corn starch
2 g flavours
800 mg colour
200 mg BanLec

Production

30 g gum base are poured into a double stage pot and warmed up with indirect steaming to approximately 100° C. Special care is needed not to introduce any water in the mixture. The mixture is stirred until it is warm and gooey. Instead of indirect steaming, one can use microwavable pot and warm up for approximately 30 sec at 115° C. until it is warm and gooey.

30 g corn syrup are poured into a clean double stage indirect steaming pot and warmed up to make it a smooth liquid, alternatively corn syrup is warmed up for 25 sec inside a microwave at 120° C.

The gooey gum base and liquified corn syrup are mixed and 2 g flavours, 800 mg of a desired colour are added, Mixing is continued for 5 minutes to form a uniform smooth mixture.

27 g of corn starch powder are added to the mixture and mixing is continued for 10 minutes. Subsequently, the mixture is mixed and cooled down in a two-stage pot with water cooling outside until reaching to 35° C. Parallel to the previous step, 10 g powder corn starch are mixed with 200 mg BanLec to a homogenic powder mixture.

Once the mixtures of gum base and corn starch is cooled down to 35° C., the powder mixture of corn starch and BanLec is added. Kneading is continued for 10 minutes to form the chewing gum composition.

The composition can be formed and cut to small pieces 70 pieces of 1.43 g per piece are provided. e Each piece contains 2.86 mg (2860 μg) of BanLec.

The mass balance equations below show that using one piece of chewing gum weighing 1.43 g that contains 2.86 mg BanLec for 15 minutes, can activate mouth media with a sufficient amount of free BanLec to protect the host exposed to a highly virus contaminated environment.

A first amount of MBL(BanLec) binds to virus particles. A second amount of MBL(BanLec) is bound to the mannose or oligosaccharides inside the chewing gum. A third amount of MBL(BanLec) is bound to polysaccharides such as starches that are released during chewing by the effect of amylase enzyme in salvia. A fourth amount of 4-MBL(BanLec) is swallowed. A fifth amount of MBL(BanLec) remains in the mouth media due to surface layer generated. A sixth amount of MBL(BanLec) is unbound or free MBL in mouth media.

For an effective inhibition the following equations apply:

$${\dot{m}}_{t} = {\dot{m}}_{1} + {\dot{m}}_{2} + ⋯ {\dot{m}}_{6} = {\sum }_{i = 1}^{6} {\dot{m}}_{i}$$

\dot{m}₁ is a function of time and virus contamination in the media that a host is exposed to.

\dot{m}₁=f(Nᵥᵢᵣᵤₛₑₛ,δt)

\dot{m}₂ is a function of oligosaccharides and mannose contents in chewing gum.

\dot{m}₂=f(M_{sugar})

\dot{m}₃ is a function of time and effects of amylase enzyme on polysaccharides like starch.

\dot{m}₃=f(M_{starch},δt)

\dot{m}₄ is a function of time and swallow up.

\dot{m}₄=f(\dot{Q}_{swallow-up},δt)

(\dot{m}₅+\dot{m}₆)≥\dot{m}₁

Based on the current example

$${\dot{m}}_{1} = f ( {N}_{viruses} ( 16000 ) , δ t ( 3 hr ) ) \\ {\dot{m}}_{2} = f ( {M}_{sugar} ( \frac{30 gr}{70 piece} ) ) \\ {\dot{m}}_{3} = f ( {M}_{starch} ( \frac{37 gr}{70 piece} ) , δ t ( 3 hr ) )$$

Humans swallow between 500-700 times a day, around 3 times an hour during sleep, once per minute while awake and even more during meals.

\dot{m}_{4,min}=f(\dot{Q}_{swallow-up,min}(3×3×100 ml),δt(3 hr))

\dot{m}_{4,max}=f(\dot{Q}_{swallow-up,max}(1×3×60×100 ml),δt(3 hr))

### Example 3: Throat Lozenges

Throat lozenges are prepared according to the following procedure: As basic ingredients, carbohydrate sweeteners can be used. Alternative sweeteners, such as isomalt may also be used. Additionally, either zinc gluconate glycine, zinc acetate or pectin can be added as an oral demulcent. Mannose-binding lectin is added to this basis.

The person skilled in the art is familiar with many other ingredients that can be added to throat lozenges such as: tart green apple extract, glycerol, manuka honey, aloe vera, American ginseng, lysozyme, and orchid extract. Lozenges may also contain benzocaine, an anaesthetic, or eucalyptus oil. Optional additives such as dextromethorphan can be used as well.

The moisture content and weight of hard candy lozenge should be between, 0.5 to 1.5% and 1.5 to 4.5 g respectively. Depending on the weight per piece, the content of mannose-binding lectin varies. In this example it was 0.1 to 0.5 wt % of the composition. However, more or less than that are also possible.

Table 4 lists ingredients for throat lozenges:

TABLE 4

| | wt % of the | |
|---|---|---|
| ingredients | composition | example and or functionality |
| benzocaine | 7.75% | Is an ester local anaesthetic commonly |
| (optional) | (up to 20%) | used as a topical pain reliever or in |
| | | cough drops. |
| menthol | | |
| (optional) | | |
| Eucalyptus oil | 3.1 mg | Eucalyptus oil is the generic name for |
| | | distilled oil from the leaf of eucalyptus, |
| | | a genus of the plant family myrtaceae |
| | | native to Australia and cultivated |
| | | worldwide. |
| zinc gluconate | | Zinc gluconate is the zinc salt of gluconic |
| glycine | | acid. It is an ionic compound consisting of |
| | | two anions of gluconate for each zinc(II) |
| | | cation. Zinc gluconate is a popular form for |
| | | the delivery of zinc as a dietary |
| | | supplement. |
| | | Zinc gluconate has been used in lozenges |
| | | for treating the common cold. |
| | | Alternatively, zinc acetate can be used |
| | | that has been found to have a greater |
| | | effect on the duration of colds. |
| pectin | | Demulcent; A demulcent is an agent that |
| | | forms a soothing film over a mucous |
| | | membrane, relieving minor pain and |
| | | inflammation of the membrane. |
| | | Demulcents are sometimes referred to as |
| | | mucoprotective agents. Demulcents such |
| | | as pectin, glycerine, honey, and syrup are |
| | | common ingredients in cough mixtures |
| | | and cough drops. Methylcellulose, |
| | | propylene glycol, and glycerine are |
| | | synthetic demulcents. |
| dextromethorphan | | |
| sweeteners | | e.g. natural honey (additional effect as |
| | | demulcent) |
| flavours | | Lemon, orange, etc |
| Mannose-binding | 0.1-0.5% | BanLec or another mannose-binding |
| lectin | | lectin, e.g. human MBL |

For production of the throat lozenges mannose-binding lectin solution is mixed with the base ingredients, when the temperature of the base mixture is 38-40° C. Mannose-binding lectin solution is added and mixing continues for 10 minutes. A throat lozenge can be applied twice a day to support the immune system.

Recipe to Prepare Throat Lozenges

Ingredients
1 cup of sugar
½ cup of water
1 tablespoon of lemon juice
1 tablespoon of honey
½ teaspoon of ground ginger
¼ teaspoon of ground cloves
10 g powder corn starch
200 mg BanLec

Production

Sugar and water are added into a pot, a tablespoon of lemon juice is added into the pot. The lemon juice will provide some vitamin C. Honey, ginger and ground cloves are added into the mixture.

The honey is antibacterial, soothes the throat, and is a cough suppressant. The ginger helps maintain the immune system, is an anti-inflammatory, helps with pain relief, and also alleviates of nausea. Ground cloves reduce phlegm, and are also a source of antioxidants. The ingredients are heated and stirred together. Once simmering the mixture is stirred regularly for 15 to 20 minutes. Subsequently, the mixture is left to cool until the liquid is thick and syrupy. Little dots are poured on parchment paper to form the lozenges.

Parallel to the previous step, sugar, 10 g powder corn starch and 200 mg BanLec are mixed to form a completely homogenic powder mixture. The lozenges are evenly covered with the powdered mixture.

### Example 4: Toothpaste

For this example, a toothpaste as generally known by a person skilled in the art was used. Additives can be added, such as: fluoride, glycerol, sorbitol, calcium carbonate, sodium lauryl sulphate. Additionally, mannose-binding lectin is added in this example.

The content of mannose-binding lectin varies, from 0.1 to 0.5 wt %. More or less than that can be added in another embodiment.

Table 5 lists ingredients for the toothpaste.

TABLE 5

| | wt % of the | |
|---|---|---|
| ingredient | composition | example and or functionality |
| abrasives | at least 50% | These insoluble particles help remove plaque from |
| | | the teeth. The removal of plaque and calculus |
| | | prevents the accumulation of tartar and is widely |
| | | claimed to help minimize cavities and periodontal |
| | | disease. |
| | | Representative abrasives include particles of |
| | | aluminum hydroxide (Al(OH)3), calcium carbonate |
| | | (CaCO3), various calcium hydrogen phosphates, |
| | | various silicas and zeolites, and hydroxyapatite |
| | | (Ca5(PO4)3OH). |
| fluoride | 1000-1450 ppm | Fluoride in various forms is the most popular active |
| | or | ingredient in toothpaste to prevent cavities. The |
| | 0.312% w/w | additional fluoride in toothpaste has beneficial |
| | | effects on the formation of dental enamel and |
| | | bones. Sodium fluoride (NaF) is the most common |
| | | source of fluoride, but stannous fluoride (SnF2), |
| | | olaflur (an organic salt of fluoride), and sodium |
| | | monofluorophosphate (Na2PO3F) are also used. |
| | | Stannous fluoride has been shown to be more |
| | | effective than sodium fluoride in reducing the |
| | | incidence of dental caries and controlling gingivitis |
| | | but causes somewhat more surface stains. |
| surfactants | | Many, although not all, toothpastes contain sodium |
| or | | lauryl sulfate (SLS) or related surfactants |
| detergents | | (detergents). SLS is found in many other personal |
| | | care products as well, such as shampoo, and is |
| | | mainly a foaming agent, which enables uniform |
| | | distribution of toothpaste, improving its cleansing |
| | | power. |
| water | 20-42% | |
| antibacterial | | Triclosan, an antibacterial agent, is a common |
| agents | | toothpaste ingredient in the United Kingdom. |
| | | Triclosan or zinc chloride prevent gingivitis and, |
| | | according to the American Dental Association, |
| | | helps reduce tartar and bad breath. |
| flavour | | The three most common flavourings are |
| | | peppermint, spearmint, and wintergreen. |
| | | Toothpaste flavoured with peppermint-anise oil is |
| | | popular in the Mediterranean region. These |
| | | flavours are provided by the respective oils, e.g. |
| | | peppermint oil. More exotic flavours include |
| | | anethole anise, apricot, bubblegum, cinnamon, |
| | | fennel, lavender, neem, ginger, vanilla, lemon, |
| | | orange, and pine. Alternatively, unflavoured |
| | | toothpastes can be provided. |
| remineralizers | | Hydroxyapatite nanocrystals and a variety of |
| | | calcium phosphates are included in formulations |
| | | for remineralization, i.e. the reformation of enamel. |
| miscellaneous | | Agents are added to suppress the tendency of |
| components | | toothpaste to dry into a powder. Included are |
| | | various sugar alcohols, such as glycerol, sorbitol, |
| | | or xylitol, or related derivatives, such as 1,2- |
| | | propylene glycol and polyethyleneglycol. Strontium |
| | | chloride or potassium nitrate is included in some |
| | | toothpastes to reduce tooth sensitivity. |
| mannose- | 0.1-0.5% | BanLec or another mannose-binding lectin, e.g |
| binding lectin | | human MBL |

When the temperature of the base mixture is appropriate (lower than 38 or 38-40° C.), MBL powder can be added and mixing continues for 10 minutes.

At least two times toothbrush per day is recommended. The immune response can be enhanced.

Recipe to Prepare Toothpaste

Ingredients
½ cup coconut oil
2-3 TBSP baking soda
2 small packets stevia powder
15-20 drops peppermint essential oil (or cinnamon essential oil)
10 drops myrrh essential oil (optional)
10 g powder corn starch
200 mg BanLec

Production

Coconut oil is melted and baking soda and stevia powder are added. The mixture is cooled while mixing is continued and the essential oil is added. Parallel to the previous step powder corn starch and 200 mg BanLec are mixed to form a homogenic powder mixture. The powder mixture is added to the cooled mixture to provide a toothpaste composition.

### Example 5: Mouthwash or Gargling Liquid

In this example, a mouthwash or gargling liquid is prepared. The basic ingredients are known to the person skilled in the art. A mouthwash or gargling liquid may contain alcohol, benzydamine, benzoic acid, betamethasone, cetylpyridinium chloride (antiseptic, antimalodor), chlorhexidine digluconate and hexetidine (antiseptic), edible oils, essential oils and phenols. The present composition additionally comprises a mannose-binding lectin.

The content of mannose-binding lectin varies, in this example from 0.1 to 0.5 wt %. In another embodiment more or less than that can be comprised.

Table 6 lists ingredients for mouthwash/gargling solution

| | wt % of the | |
|---|---|---|
| Ingredient | composition | example and or functionality |
| alcohol | | Alcohol is added to mouthwash not only to destroy |
| | | pathogens, e.g. bacteria, but to act as a carrier |
| | | agent for essential active ingredients such as |
| | | menthol, eucalyptol and thymol, which help to |
| | | penetrate plaque. |
| benzydamine | | Analgesics; |
| | | In painful oral conditions such as aphthous |
| | | stomatitis, analgesic mouth rinses are sometimes |
| | | used to ease pain, commonly used before meals to |
| | | reduce discomfort while eating. |
| benzoic acid | | Acts as a buffer |
| betamethasone | | Betamethasone is sometimes used as an anti- |
| | | inflammatory, corticosteroid mouthwash. It may be |
| | | used for severe inflammatory conditions of the oral |
| | | mucosa such as the severe forms of aphthous |
| | | stomatitis. |
| cetylpyridinium | 0.05% | Antiseptic, antimalodor; |
| chloride | | Cetylpyridinium chloride containing mouthwash is |
| | | used in some specialized mouthwashes for |
| | | halitosis. |
| chlorhexidine | 0.12-0.2% | Antiseptic; |
| digluconate and | | Chlorhexidine digluconate is a chemical antiseptic. |
| hexetidine | | It has anti-plaque action, but also some anti-fungal |
| | | action. |
| edible oils | | Phenolic compounds include essential oil |
| essential oils and | | constituents that have some antibacterial |
| phenols | | properties, like phenol, thymol, eugenol, or |
| | | eucalyptol. |
| | | Essential oils are oils are extracted from plants. |
| | | Mouthwashes based on essential oils could be |
| | | more effective than traditional mouthcare-for anti- |
| | | gingival treatments. They have been found |
| | | effective in reducing halitosis and are being used |
| | | in several commercial mouthwashes. |
| fluoride | | Prevention of cavities |
| flavouring agents | | |
| and xylitol | | |
| Hydrogen | 1.5% | Hydrogen peroxide can be used as an oxidizing |
| peroxide | | mouthwash. It kills anaerobic bacteria, and also |
| | | has a mechanical cleansing action when it froths |
| | | as it comes into contact with debris in mouth. |
| lactoperoxidase | | Saliva substitute; |
| | | Enzymes and proteins such as lactoperoxidase, |
| | | lysozyme, lactoferrin have been used in |
| | | mouthrinses (e.g. Biotene) to reduce oral bacteria |
| | | and hence the acid produced by bacteria. |
| Lidocaine | | Lidocaine is useful for the treatment of mucositis |
| | | symptoms (inflammation of mucous membranes) |
| | | that is induced by radiation or chemotherapy. |
| methyl salicylate | | Methyl salicylate functions as an anti-septic, anti- |
| | | inflammatory, analgesic, flavouring, and fragrance. |
| | | Methyl salicylate has some anti-plaque action, but |
| | | less than chlorhexidine. Methyl salicylate does not |
| | | stain teeth. |
| nystatin | | Nystatin suspension is an antifungal ingredient |
| | | used for the treatment of oral candidiasis. |
| potassium oxalate | | A randomized clinical trial found promising results |
| | | in controlling and reducing dentine hypersensitivity |
| | | when potassium oxalate mouthrinse was used in |
| | | conjugation with toothbrushing. |
| sanguinarine | | Sanguinarine-containing mouthwashes are |
| | | marketed as anti-plaque and anti-malodor. |
| sodium | | Sodium bicarbonate is sometimes combined with |
| bicarbonate | | salt to make a simple homemade mouthwash, |
| | | indicated for any of the reasons that a salt water |
| | | mouthwash might be used. Pre-mixed |
| | | mouthwashes of 1% sodium bicarbonate and |
| | | 1.5% sodium chloride in aqueous solution are |
| | | marketed. |
| sodium chloride | | Salt water mouth wash is made by dissolving 0.5 |
| | | to 1 teaspoon of table salt into a cup of water, |
| sodium lauryl | | Foaming agent; |
| sulfate | | Sodium lauryl sulfate (SLS) is used as a foaming |
| | | agent in many oral hygiene products including |
| | | many mouthwashes. Some may suggest that it is |
| | | probably advisable to use mouthwash at least an |
| | | hour after brushing with toothpaste when the |
| | | toothpaste contains SLS, since the anionic |
| | | compounds in the SLS toothpaste can deactivate |
| | | cationic agents present in the mouth rinse. |
| sucralfate | | Sucralfate is a mucosal coating agent, composed |
| | | of an aluminum salt of sulphated sucrose. |
| tetracycline | | Antibiotic; |
| | | Tetracycline is an antibiotic which may sometimes |
| | | be used as a mouthwash in adults. It is sometimes |
| | | use for herpetiforme. |
| tranexamic acid | | 4.8% tranexamic acid solution is sometimes used |
| | | as an antifibrinolytic mouthwash to prevent |
| | | bleeding during and after oral surgery in persons |
| | | with coagulopathies (clotting disorders) or who are |
| | | taking anticoagulants (blood thinners such as |
| | | warfarin) |
| triclosan | | Triclosan is a non-ionic chlorinate bisphenol |
| | | antiseptic found in some mouthwashes. When |
| | | used in mouthwash (e.g. 0.03%), there is moderate |
| | | substantivity, broad spectrum anti-bacterial action, |
| | | some anti-fungal action and significant anti-plaque |
| | | effect, especially when combined with copolymer |
| | | or zinc citrate. Triclosan does not cause staining of |
| | | the teeth. |
| Zinc chloride | | Astringents like zinc chloride provide a pleasant- |
| | | tasting sensation and shrink tissues. Zinc when |
| | | used in combination with other anti-septic agents |
| | | can limit the build-up of tartar. |
| Mannose-binding | 0.1-0.5% | BanLec or another mannose-binding lectin, e.g |
| lectin | | human MBL |

When the temperature of the base mixture is appropriate (lower than 38 or 38-40° C.), mannose-binding lectin solution is added and mixing continues for 10 minutes.

Two times gargling per day is recommended. The immune system can be supported to tackle infectious diseases.

Gargling Solution:

Ingredients
100 ml warm water
2 g of salt
1 g powder corn starch
200 mg BanLec

Production

Salt is dissolved in warm water. Parallel to the previous step, 1 g powder corn starch is mixed with 200 mg BanLec to form a homogenic powder mixture. The powder mixture is dissolved inside the cooled solution to provide a gargling composition.

### Example 6: Nasal and or Oral Drops

In this example nasal and or oral drop was prepared. Basic ingredients are known to a person skilled in the art. In this example, water, and sodium chloride are used. Additionally, mannose-binding lectin is added. The concentration of mannose-binding lectin varies in this example from 0.1 to 0.5 wt %. In another embodiment more or less than that can be comprised.

Table 7 lists ingredients for nasal and or oral drops

| | wt % of the | |
|---|---|---|
| ingredient | composition | example and or functionality |
| Water | | This is the main ingredient |
| sodium | | hypertonic (3% sodium chloride or sea water), isotonic |
| chloride | | (0.9% sodium chloride) and hypotonic (0.65% sodium |
| (optional: | | chloride). |
| e.g. saline | | Isotonic solutions have the same salt concentration as |
| sprays) | | the human body, whereas hypertonic solutions have a |
| | | higher salt content and hypotonic solutions have a |
| | | lower salt content. |
| plant- | | eucalyptus, ginger, capsaicin, tea-tree oil |
| derived | | |
| ingredients | | |
| (optional) | | |
| mannose- | 0.1-0.5% | BanLec or another mannose-binding lectin, e.g. human |
| binding lectin | | MBL |

When the temperature of the base mixture is appropriate, mannose-binding lectin solution is added, and mixing continues for 10 minutes. The 10 drops of this mixture can be used every 3 hours.

Recipe to prepare Nasal and Oral drops

Ingredients
100 ml purified water
1 g sea salt
0.5 g baking soda
1 g powder corn starch
200 mg BanLec

Production

The water is heated and sea salt and baking soda are added. The mixture is stirred until the solution is completely dissolved. Then the solution is left to cool. Once the solution is created, remove the pot from the stove, and let the solution cool. Parallel to the previous step, 1 g powder corn starch are mixed with 200 mg BanLec to form a homogenic powder mixture. The powder mixture is dissolved in the cooled solution to provide a nasal and or oral drops composition.

### Example 7: Aerosol Spray

The oral and/or nasal spray can be used at the point of entry into the body for prophylaxis or treatment of infectious diseases, particularly respiratory diseases, such as COVID-19.

Inhalation solution and suspension products are aqueous-based formulations that contain active ingredients and can also contain additional excipients. Aqueous-based oral inhalation solutions and suspensions must be sterile. Inhalation solutions and suspensions are intended for delivery to the lungs by inhalation for local and/or systemic effects and are to be used with a nebulizer.

The products contain active ingredients and can also contain additional excipients (viscosity modifiers, emulsifiers, buffering agents). MBL(BanLec) is one of the main active ingredients in the present formulation

The use of preservatives or stabilizing agents in inhalation spray formulations is discouraged and is the same here. The solution should be stored in special inhalators.

The dose is delivered by the integral pump components of the inhaler to the lungs by oral inhalation for local and/or systemic effects. The inhaler comprises a container, closure, and pump.

Metered dose inhalers for inhalation spray products operate on the basis of mechanical or power assistance and/or energy from the patient's inspiration to produce the aerosol.

Ingredients
100 ml water (ad injectionem)
0.9 g NaCl
buffer
150 mg BanLec
0.5 g essence (optional)

Production

Water is poured into a sterile container and BanLec and NaCl are added. The solution is mixed and during mixing buffer (e.g. HEPES) is added. The pH-value is adjusted to 7.5. If desired, an essence can be added to the mixture. The mixture is filled into an inhaler that can nebulize the mixture to provide an aerosol spray.

### Example 8: Eye Drops

Ingredients
100 ml water (ad injectionem)
0.9 g Na Cl
Buffer
150 mg BanLec
0.4 g polyethylene glycol 400
0.3 g propylene glycol

Production

Water is poured into a sterile container and BanLec, NaCl, polyethylene glycol, and propylene glycol are added. The solution is mixed and buffer (e.g. HEPES) is added during mixing to adjust the pH-value to 7.5.

### Example 9: Ice Cream

| Cream ice cream | % | Fruit sorbet vegan | % |
|---|---|---|---|
| Whole milk | 60 | Fruit pulp (e.g. strawberry) | 50 |
| Cream 32% fat | 9.2 | Water | 21.2 |
| Egg pasteurized | 6 | Sugar | 20 |
| Sugar | 15 | Glucose syrup | 2 |
| Skimmed milk powder | 3 | Lemon juice | 2 |
| Dextrose | 2.5 | Dextrose | 2.5 |
| Inulin | 1.5 | Inulin | 1.5 |
| Glucose syrup | 2 | Salt | 0.1 |
| Salt | 0.1 | Guar gum | 0.2 |
| Guar gum | 0.2 | Locust bean gum | 0.4 |
| Locust bean gum | 0.4 | | |
| MBL (BanLec) | 0.1 | MBL (BanLec) | 0.1 |
| Total | 100 | Total | 100 |

Possible Further Ingredients:
Invert sugar
Cane sugar
Baobab
Fructose
Emulsifier E471
Citric acid
Milk substitute
Maltodextrin
various binders
Flavour defining
ingredients:
Vanilla
Nut pulp
various fruits
Coffee
Cocoa

Experiments

For all experiments a stock solution of 2.2 E+06 PFU/mL of Human 2019-nCoV Isolate is used and various viral working stocks are grown in Vero CCL81 cells using fetal calf serum (FCS)-free cell culture medium (OptiPro from Gibco). The working stock aliquots used in the experiment are the virus passage (VP) 2 with a PFU/ml of 1.74 E+04.

Vero-cells CCL81 (3 E+04 cells/well in serum free Gibco OptiPro) are seeded into 48 well plates 24 hrs. prior to infection. The virus is stored at −80° C. with cells. To purify the virus suspension is centrifuged for 1 min at 13.000 rpm. The cell pellet stays in the vial and the pure virus supernatant is used for the experiment. The virus is pre-incubated with the substance for 1 hr at 37° C. with 5% CO2. With 200 μl of the virus substance mix, there is a final virus load of 2 μl virus on the cells for the infection. The plate is incubated for 60 min at 37° C. with 5% CO2.

1-hour post infection the cells are washed two times with PBS and covered with 440 μl fresh pre-warmed cell culture medium (Gibco OptiPro).

After 10 minutes of incubation at RT 140 μl from the cell culture medium supernatant is removed to determine the starting concentration of viral copy numbers (t=0 h). Constant Ct-values are reached around 27 after the two washing steps. The samples are stored under −80° C. After 48 hrs. of incubation again under previous conditions (37° C. with 5% CO2), further 140 μl of cell culture medium supernatant is obtained (t=48 h) and RNA is isolated immediately to determine virus copy numbers.

The experiments include internal controls for the efficiency of infection. Cells infected with virus without any substance addition (positive control) and cells not infected with virus (negative control). Both were handled the same as the substance samples in the view of dilution, time, conditions and earning supernatant for further treatment.

Viral RNA was isolated from cell culture medium supernatant by using QIAamp® Viral RNA Mini Kit, as recommended by CDC.

The RT-qPCR, to detect the viral load of the samples, was performed based on the CDC recommendation using QuantiTect Multiplex RT-PCR Kit with a Rotor Gene Q cycler:

2019-nCoV_N1-F 2019-nCoV_N1 Forward Primer
5′-GAC CCC AAA ATC AGC GAA AT-3′
2019-nCoV_N1-R 2019-nCoV_N1 Reverse Primer
5-TCT GGT TAC TGC CAG TTG AAT CTG-3′
2019-nCoV_N1-P 2019-nCoV_N1 Probe
5′-FAM-ACC CCG CAT TAC GTT TGG TGG ACC-BHQ1-3′
FAM, BHQ-1

All Ct-values higher than 40 are considered negative and undetermined. Virus replication was assessed by comparing Ct values after infection (t=0 h) with Ct values after different time-periods of culturing. If there is a difference of minimum 4 cycles (t=48 is higher) we can see an inhibition of the virus replication in the cells.

Test 1

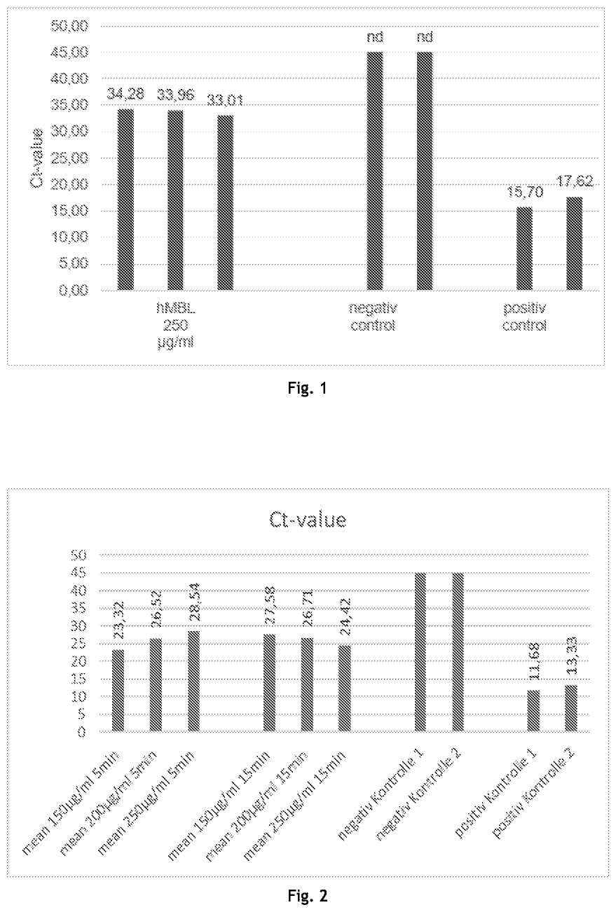

A virus neutralization assay is performed following the experimental procedures as described above using human MBL (hMBL) at a concentration of 0.5 mg/ml. Commercially available SARS-CoV-2 is incubated with commercially available human MBL. The hMBL is used in a dilution with a final concentration of 250 μg/ml. Target cells (Vero, primate) are incubated with the mixture of hMBL and SARS-CoV-2. Virus concentration in the cell culture supernatant is determined by SARS-CoV-2 qRT-PCR at 48 hrs after infection. As positive control SARS-CoV-2 without MBL pre-treatment is used and as negative control a sham sample is used. After 48 h the supernatant is removed and analysed for virus-RNA. The hMBL neutralization experiment was performed in three technical replicates. The results are shown in FIG. 1. The qRT-PCR results showed in the triplicates with hMBL viral RNA concentration in the cell culture supernatant a mean of 33.75 cycles (Ct value) with a standard deviation of 0.659. Compared to the positive controls (duplicates), which are infected with the same amount of virus but without hMBL-pre-incubation yielded Ct values of 15.7 and 17.62, respectively. Based on the difference of Ct values between positive controls (mean Ct 16.66) and hMBL-inactivated tests (mean Ct 33.75) a 1,396*105-fold reduction of virus load by hMBL can be calculated. Virus-RNA-levels in the test sample are only marginally higher than in the sham sample, while in the positive control sample RNA-levels are considerably higher. It is shown that recombinant, human MBL is able to inhibit infection of target cells.

Test 2

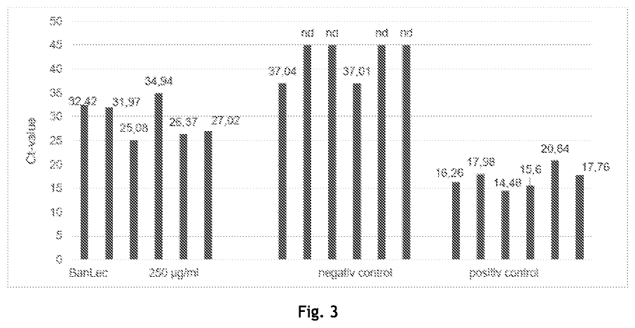

Commercially available SARS-CoV-2 is incubated with 150 μg/ml, 200 μg/ml, and 250 μg/ml of commercially available recombinant BanLec (Banana lectin). Target cells (Vero, primate) are incubated with the mixture of MBL and SARS-CoV-2 for 5 minutes, 15 minutes, 30 minutes and 1 hour. Each experiment is carried out three times. As positive control SARS-CoV-2 without MBL pre-treatment is used and as negative control a sham sample is used. After 1 hour the supernatant is removed and analysed for virus-RNA. The results of the mean value after 5 min and 15 min of 150 μg/ml, 200 μg/ml, and 250 μg/ml is shown in FIG. 2. Virus-RNA-levels in the test sample is only marginally higher than in the sham sample, while in the positive control sample RNA-levels is considerably higher. It is shown that recombinant BanLec is able to inhibit infection of target cells (Vero, primate) even within a very short time frame very efficiently.

Test 3

The experimental conditions are as described in test 2. 2 μl virus suspension corresponds to a MOI=0.002 and is used in each well for infection. Virus neutralizing activity of 250 μg/ml BanLec with 1 hr pre-incubation time is evaluated. SARS-CoV-2 RNA Ct values are determined in cell culture supernatant by qRT-PCR at 48 hrs after infection. The results are shown in FIG. 3. The qRT-PCR results in six technical replicas with BanLec in mean Ct values 29.63. Compared to the six positive controls with a mean Ct value of 17.15 an approximately 5.8*103-fold reduction in virus concentration is achieved.

### SEQUENCE LISTING

<210> SEQ ID NO:

<211> 227

<212> PRT

<213> human

<400>

Glu Thr Val Thr Cys Glu Asp Ala Gln Lys Thr Cys Pro Ala Val Ile

1 5 10 15

Ala Cys Ser Ser Pro Gly Ile Asn Phe Pro Gly Lys Asp Gly Arg
20 25 30

Asp Gly Thr Lys Gly Glu Lys Gly Glu Pro Gly Gln Gly Leu Arg Gly
35 40 45

Leu Gln Gly Pro Pro Gly Lys Leu Gly Pro Pro Gly Asn Pro Gly Pro
50 55 60

Ser Gly Ser Pro Gly Pro Lys Gly Gln Lys Gly Asp Pro Gly Lys Ser

65 70 75 80

Pro Asp gly Asp Ser Leu Ala Ala Ser Glu Arg Lys Ala Leu Gln
85 90 95

Thr Glu Met Arg Ile Lys Lys Trp Leu Thr Phe Ser Leu Gly Lys
100 105 110

Gln Val Gly Asn Lys Phe Phe Leu Thr Asn Gly Glu Ile Met Thr Phe
115 120 125

Glu Lys Val Lys Ala Leu Cys Val Lys Phe Gln Ala Ser Val Ala Thr
130 135 140

Pro Arg Asn Ala Ala Glu Asn Gly Ala Ile Gln Asn Leu Ile Lys Glu
145 150 155

Glu Ala Phe Leu Gly Ile Thr Asp Glu Lys Thr Glu Gly Gln Phe Val

160 165 170 175

Asp Leu Thr Gly Asn Arg Leu Thr Tyr Thr Asn Trp Asn Glu Gly Glu
180 185 190

Pro Asn Asn Ala Gly Ser Asp Glu Asp Cys Val Leu Leo Leu Lys Asn
195 200 205

Gly Gln Trp Asn Asp Val Pro Cys Ser Thr Ser His Leu Leu Ala Val Cys
210 215 220

Glu Phe Pro ile
225

<210> SEQ ID NO: 2

<211> 112

<212> PRT

<213> human

<400>

Val Gly Asn Lys Phe Phe Leu Thr Asn Gly Glu Ile Met Thr

1 5 10

Phe Glu Lys Val Lys Ala Leu Cys Val Lys Phe Gln Ala Ser

15 20 25

Val Ala Thr Pro Arg Asn Ala Ala Glu Asn Gly Ala Ile Gln
30 35 40

Asn Leu Ile Lys Glu Glu Ala Phe Leu Gly Ile Thr Asp Glu
45 50 55

Lys Thr Glu Gly Gln Phe Val Asp Leu Thr Gly Asn Arg Leu
60 65 70

Thr Tyr Thr Asn Trp Asn Glu Gly Glu Pro Asn Asn Ala Gly
75 80

Ser Asp Glu Asp Cys Val Leu Leu Leu Lys Asn Gly Gln Trp

85 90 95

Asn Asp Val Pro Cys Ser Thr Per His Leu Ala Val Cys Glu
100 105 110

<210> SEQ ID NO: 3

<211> 17

<212> PRT

<213> human

<400>

Glu Arg Lys Ala Leu Gln Thr Glu Met Ala Arg Ile Lys Lys

1 5 10

Trp Leu Thr Phe
15

1. A method of performing prophylaxis and/or treatment of an infectious diseases, comprising the step of locally administering mannose-binding lectin (MBL), or portions thereof, to an individual.

2. The method of claim 1, wherein the mannose-binding lectin is administered orally, nasally, or to a mouth, nose, lung, throat, or eye of the individual.

3. The method of claim 1, wherein the mannose-binding lectin is administered by means of a chewing gum, ice cream, a lozenge, a toothpaste, a mouth wash and/or a gargling solution, a nasal spray, nasal drops, a nasal cream, eye drops, an eye cream, or via inhalation or an aerosol spray.

4. The method of claim 1, wherein the mannose-binding lectin is human MBL corresponding to SEQ ID NO:1, particularly recombinant human MBL, or portions thereof, particularly corresponding to SEQ ID NO:2, more particularly corresponding to SEQ ID NO:3.

5. The method of claim 1, wherein the mannose-binding lectin is a plant-derived mannose binding lectin selected from the group consisting of ACA (Allium cepa), APA (Allium porrum), ASA I (Allium sativum), ASA II (Allium sativum), AUA (Allium ursinum), ArtinM (Artocarpus heterophyllus), B7U6V0 (Zingiber officinalis), BanLec (Musa acuminate), ConA (Canavalia ensiformis), DB1 (Dioscorea batatas), LEA (Solanum lycopersicum), Morniga M II (Morus nigra) or Q1 S2H7 (Curcuma zedoria), and GRFT (Griffithsin), or is an animal-derived mannose-binding lectin selected from the group consisting of LvCTL1 (Litopenaeus vannamei), Pl-MBL (Pacifastacus leniusculus), PcLec4 (Pacifastacus clarkia), trout-MBL1 (Oncorhynchus mykiss), trout-MBL2 (Oncorhynchus mykiss) and AbMb (Agaricus bisporus).

6. (canceled)

7. The method of claim 1, wherein the infectious disease is transmitted by the respiratory route.

8. The method of claim 1, wherein the infectious disease is caused by a virus, a bacterium, or a fungus.

9. The method of claim 1, wherein the infectious disease is a Coronaviridae-induced disease, particularly COVID-19.

10. The method of claim 1, wherein the mannose-binding lectin is applied at a concentration of 150 μg/ml or more.

11. The method of claim 1, wherein the mannose-binding lectin is applied every 1 to 24 hours.

12. The method of claim 1, wherein the application of the mannose-binding lectin is started 0 to 60 minutes, particularly 5 to 60 minutes, prior to a presumed exposure to a pathogen and/or wherein the application of the mannose-binding lectin is started 0 to 60 minutes, particularly 5 to 60 minutes, before a gathering with other persons.

13. A composition for use in prophylaxis and/or treatment of infectious diseases, wherein the composition comprises mannose-binding lectin (MBL), or portions thereof, and wherein the composition is adapted for local administration.

14. The composition of claim 13, wherein the composition is a chewing gum, ice cream, a lozenge, a toothpaste, a mouth wash, a gargling solution, a nasal spray, nasal drops, a nasal cream, eye drops, an eye cream or an inhalation preparation.

15. The composition of claim 13, wherein the concentration of mannose-binding lectin is 0.1 to 0.5 wt % of the composition.

16. The composition of claim 13, wherein the mannose-binding lectin has a concentration of 150 μg/ml or more.

17-20. (canceled)

21. The composition of claim 13, wherein the composition is selected from the group consisting of:
a chewing gum comprising 25 to 35 wt % of a gum base and 0.1 to 0.5 wt % mannose-binding lectin;
an ice cream comprising 0.1 to 0.5 wt % mannose-binding lectin;
a toothpaste comprising at least 50 wt % abrasives, 20 to 42 wt % water and 0.1 to 0.5 wt % mannose-binding lectin;
a lozenge comprising eucalyptus oil and 0.1 to 0.5 wt % mannose-binding lectin; and
a nasal spray comprising water and 0.1 to 0.5 wt % mannose-binding lectin.

22-25. (canceled)

26. The method of claim 1, wherein the mannose-binding lectin is combined with a pharmaceutically acceptable carrier.

27. The method of claim 1, wherein the individual is a human with MBL-deficiency.

28-32. (canceled)

33. The method of claim 1, wherein the mannose-binding lectin is applied to a face mask and the treated face mask is placed over the mouth and nose of the individual.

34. A face mask treated with mannose-binding lectin.
